(19) United States
(12) Patent Application Publication
(10) Pub. No.: US 2026/0097210 A1
(43) Pub. Date: Apr. 9, 2026

(54) IMPROVED MEASUREMENT OF EVOKED NEURAL RESPONSE CHARACTERISTICS

(71) Applicant: Saluda Medical Pty Limited, Macquarie Park, New South Wales (AU)

(72) Inventors: Daniel John Parker, Macquarie Park (AU); Samuel Nicholas Gilbert, Macquarie Park (AU); Matthew Marlon Williams, Macquarie Park (AU); Sudam Epalawattege Nimantha Dias, Macquarie Park (AU); Darayus Zarir Nanavati, Macquarie Park (AU)

(21) Appl. No.: 19/115,377

(22) PCT Filed: Oct. 2, 2023
(86) PCT No.: PCT/AU2023/050947

§ 371 Date: Mar. 26, 2025

(30) Foreign Application Priority Data
Oct. 1, 2022 (AU) 2022902847

(51) Int. Cl. A61N 1/36

(52) U.S. Cl. CPC A61N 1/36139; A61N 1/36062

(57) ABSTRACT

Disclosed is an implantable device for controllably delivering neural stimuli. The device comprises: a plurality of electrodes including one or more stimulus electrodes and one or more measurement electrodes; a stimulus source configured to provide neural stimuli to be delivered via the one or more stimulus electrodes to a neural pathway of a patient in order to evoke neural responses from the neural pathway; measurement circuitry configured to capture signal windows sensed on the neural pathway via the one or more measurement electrodes subsequent to respective neural stimuli; and a control unit configured to: control the stimulus source to provide a neural stimulus according to a stimulus intensity parameter; and measure an intensity of an evoked neural response in a signal window capture subsequent to the provided neural stimulus by correlating the captured signal window with a template. The template is orthogonal to a predetermined artefact basis that models an artefact component in the captured signal window.

The present application claims priority from Australian Provisional Patent Application No 2022902371 filed on 1 Oct. 2022, the contents of which are incorporated herein by reference in their entirety.

### TECHNICAL FIELD

The present invention relates to implantable spinal cord stimulation and in particular to improvements in the measurement of the characteristics of neural responses to stimulation.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along Aβ (A-beta) fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in stimulus artefact, which manifests in the sensed signal as a decaying output of the order of several millivolts after the end of the stimulus. As the neural response can be contemporaneous with the stimulus crosstalk and/or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Evoked neural responses are less difficult to measure when they appear later in time than the artefact, or when the signal-to-noise ratio is sufficiently high. The artefact is often restricted to a time of 1-2 ms after the stimulus and so, provided the neural response is measured after this time window, a neural response measurement can be more easily obtained. This is the case in surgical monitoring where there are large distances (e.g. more than 12 cm for nerves conducting at 60 ms⁻¹) between the stimulus and measurement electrodes so that the propagation time from the stimulus site to the measurement electrodes exceeds 2 ms, which is longer than the typical duration of stimulus artefact.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

One approach to measuring the intensity of evoked neural responses is to measure the peak-to-peak amplitude of a sensed signal, under the assumption that the extrema of the sensed signal coincide with the peaks of the neural response. Another approach to measuring intensity is to measure the root-mean-square (RMS) magnitude of the sensed signal. However, both of these measurements are subject to contamination by the artefact likely to be present in the sensed signal. In addition, even if the artefact could be first removed from the sensed signal, such measures of intensity have other disadvantages. The peak-to-peak amplitude is highly sensitive to spurious noise and is therefore a noisy measurement with frequent outliers. The RMS magnitude is non-negative and therefore has non-Gaussian noise statistics even if the sensed signal itself is subject to Gaussian noise. This complicates the further processing of the measured RMS magnitude.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are methods and devices configured to measure an intensity or other characteristic of an evoked neural response in a manner that is simultaneously accurate and robust to artefact. The disclosed technology uses a correlation detector with a template that is the residual of the projection of a signal containing a representative ECAP onto an artefact basis. The resulting correlation measure of intensity is orthogonal to any artefact that is wholly representable using the artefact basis, and accurate to the extent that the representative ECAP is representative of the actual ECAP in the sensed signal. Furthermore, the correlation measure of intensity has noise statistics that reflect the noise statistics of the sensed signal, making the measure more suitable for the subsequent processing required for both programming and operating a neuromodulation device in a closed-loop manner.

According to a first aspect of the present technology, there is provided an implantable device for controllably delivering neural stimuli. The implantable device comprising: a plurality of electrodes including one or more stimulus electrodes and one or more measurement electrodes; a stimulus source configured to provide neural stimuli to be delivered via the one or more stimulus electrodes to a neural pathway of a patient in order to evoke neural responses from the neural pathway; measurement circuitry configured to capture signal windows from signals sensed on the neural pathway via the one or more measurement electrodes subsequent to respective neural stimuli; and a control unit configured to: control the stimulus source to provide a neural stimulus according to a stimulus intensity parameter; and measure an intensity of an evoked neural response in a signal window captured subsequent to the neural stimulus by correlating the captured signal window with a template. The template is orthogonal to a predetermined artefact basis that models an artefact component in the captured signal window.

According to a second aspect of the present technology, there is provided an automated method of measuring an evoked response to neural stimuli delivered to a neural pathway of a patient. The method comprising delivering a neural stimulus to the neural pathway of the patient in order to evoke a neural response from the neural pathway, the neural stimulus being delivered according to a stimulus intensity parameter. Further, capturing a signal window sensed on the neural pathway subsequent to the delivered neural stimulus; and measuring an intensity of a neural response evoked by the delivered neural stimulus in the captured signal window by correlating the captured signal window with a template. The template is orthogonal to a predetermined artefact basis that models an artefact component in the captured signal window.

According to a third aspect of the present technology, there is provided a neural stimulation system. The system comprising: an implantable device for controllably delivering neural stimuli. The device comprising: a plurality of electrodes including one or more stimulus electrodes and one or more measurement electrodes; a stimulus source configured to provide neural stimuli to be delivered via the one or more stimulus electrodes to a neural pathway of a patient in order to evoke a neural response from the neural pathway; measurement circuitry configured to capture signal windows sensed on the neural pathway via the one or more measurement electrodes subsequent to respective neural stimuli; and a control unit configured to control the stimulus source to provide each neural stimulus according to a stimulus intensity parameter; a processor configured to: instruct the control unit to control the stimulus source to provide a neural stimulus according to a stimulus intensity parameter; and measure an intensity of an evoked neural response in a signal window captured subsequent to the provided neural stimulus by correlating the captured signal window with a template. The template is orthogonal to a predetermined artefact basis that models an artefact component in the captured signal window.

According to a fourth aspect of the present technology, there is provided a neural stimulation system comprising an implantable device for controllably delivering neural stimuli. The device comprising: a plurality of electrodes including one or more stimulus electrodes and one or more measurement electrodes; a stimulus source configured to provide neural stimuli to be delivered via the one or more stimulus electrodes to a neural pathway of a patient in order to evoke neural responses from the neural pathway; measurement circuitry configured to capture signal windows from signals sensed on the neural pathway via the one or more measurement electrodes subsequent to respective neural stimuli; and a control unit configured to: control the stimulus source to provide a neural stimulus according to a stimulus intensity parameter; and measure an intensity of an evoked neural response in a signal window captured subsequent to the neural stimulus by correlating the captured signal window with a template, wherein the template is part of clinical settings for the implantable device; and a processor configured to: derive the template to be orthogonal to a predetermined artefact basis that models an artefact component in the captured signal window; and store the template in a memory of the implantable device as part of the clinical settings for the implantable device.

According to a fifth aspect of the present technology, there is provided an automated method of programming an implantable neuromodulation device for a patient. The method comprising: deriving a template to be orthogonal to a predetermined artefact basis that models an artefact component in signal windows captured by the implantable neuromodulation device. Further, storing the template in a memory of the implantable neuromodulation device as part of clinical settings for the implantable neuromodulation device.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is a block diagram illustrating the data flow of a neural stimulation therapy system such as the system of FIG. 7;

FIG. 9 is a flow chart illustrating a method of deriving a template for a correlation-based ECAP detector such as the ECAP detector forming part of the CLNS system of FIG. 5;

FIG. 10a contains a graph showing the first five basis functions in a CPE artefact basis that may be used in the method of FIG. 9;

FIG. 10b contains a graph showing the first five basis functions in an SVD artefact basis that may be used in the method of FIG. 9;

FIG. 11 contains a graph showing the explanatory power of the SVD artefact basis obtained from a library of artefact-only sensed signals, as a function of the rank of the basis;

FIG. 12a contains graph containing a trace representing the BURD obtained from the CPE artefact basis of FIG. 10a and a trace representing the BURD obtained from a SVD artefact basis of FIG. 10b using the method of FIG. 9;

FIG. 12b contains a graph containing a trace representing the BURD obtained from using a four-lobe filter in the method of FIG. 9;

FIG. 13a contains a graph containing a set of activation plots estimated using the four-lobe filter from simulated signal windows captured at different stimulus intensities under different models of the artefact dependence on stimulus intensity;

FIG. 13b contains a graph containing a set of activation plots estimated from the same simulated signal windows as in FIG. 13a, using a BURD template derived from an SVD artefact basis; and

FIG. 14 is a flow chart illustrating a method of maintaining an effective BURD template as therapy parameters change.

### DETAILED DESCRIPTION OF THE PRESENT TECHNOLOGY

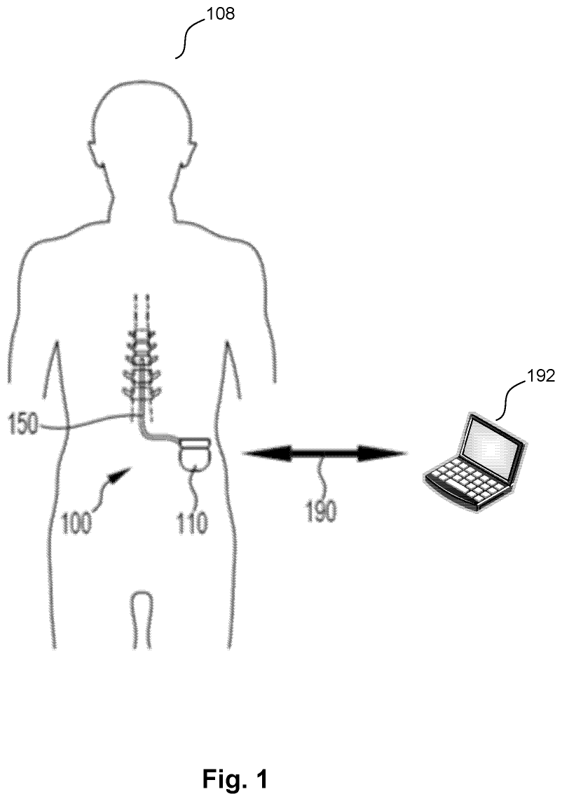

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

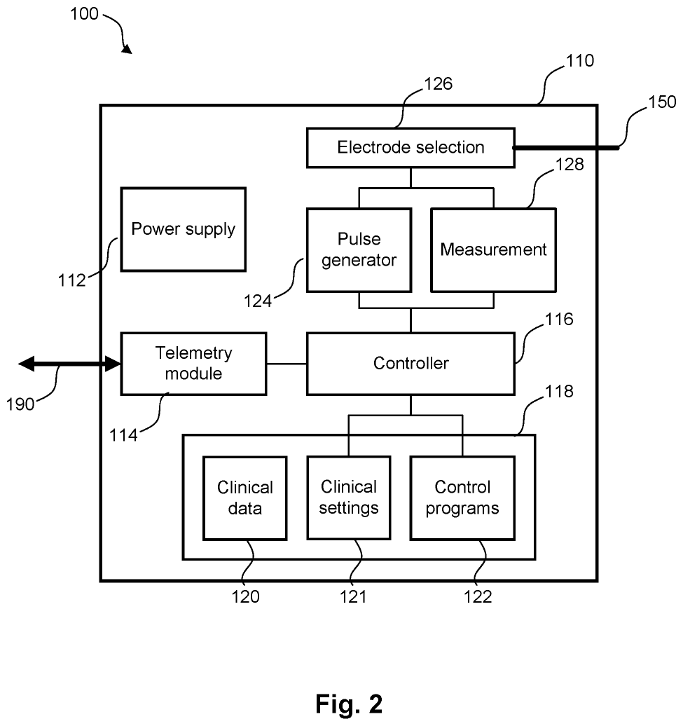

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

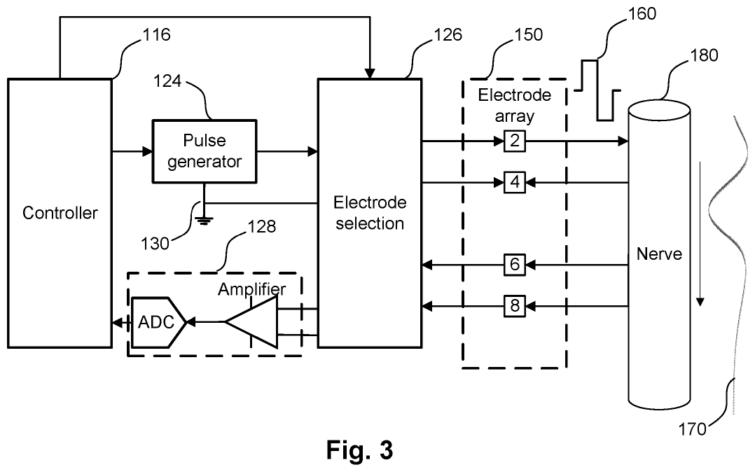

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. The set of stimulus and return electrodes and their respective polarities is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

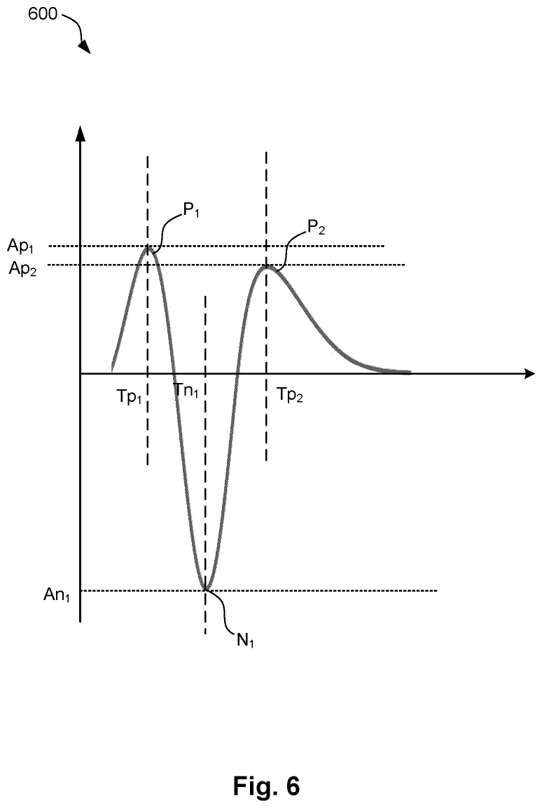

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

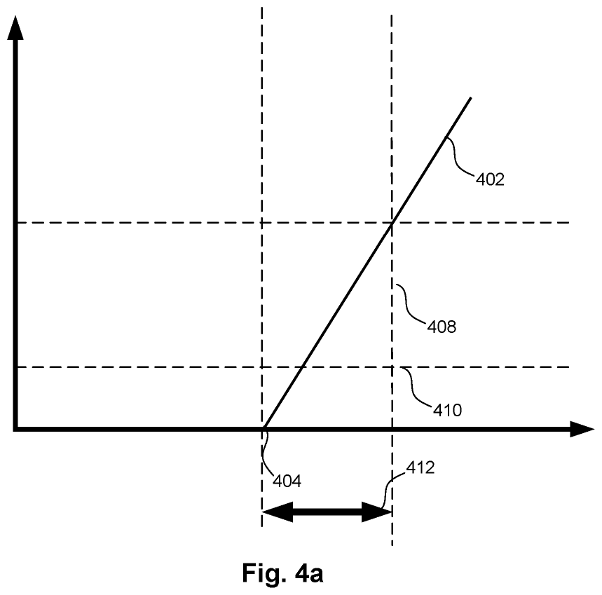

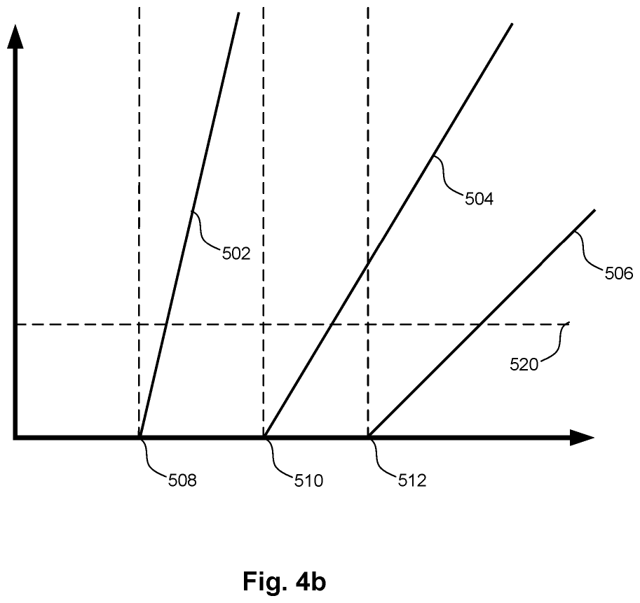

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, T is the ECAP threshold and S is the slope of the activation plot (referred to herein as the patient sensitivity). The slope S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is barely perceptible by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

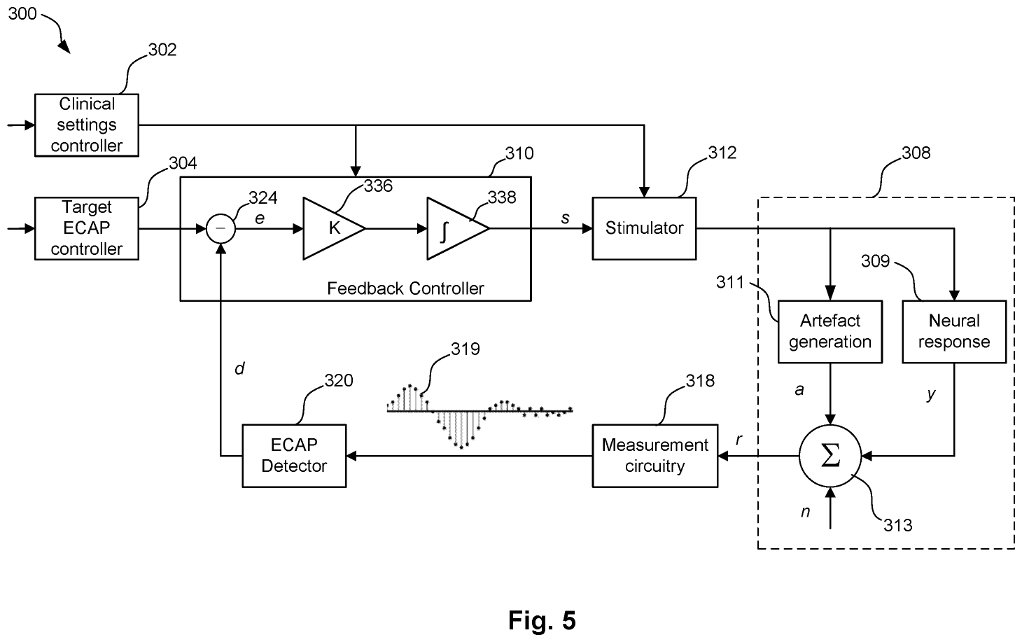

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in concert with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise), and samples the amplified sensed signal r to capture a “signal window” 319 comprising a predetermined number of samples of the amplified sensed signal r. The ECAP detector 320 processes the signal window 319 and outputs a measured neural response intensity d. In one implementation, the neural response intensity comprises a peak-to-peak ECAP amplitude. The measured response intensity d is input into the feedback controller 310. The feedback controller 310 comprises a comparator 324 that compares the measured response intensity d (an example of a feedback variable) to a target ECAP amplitude as set by the target ECAP controller 304 and provides an indication of the difference between the measured response intensity d and the target ECAP amplitude. This difference is the error value, e. The error value e is input into the feedback controller 310.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int Kedt \qquad ( 2 )$$

where K is the gain of the gain element 336 (the controller gain). This relation may also be represented as

$$δ s = Ke \qquad ( 3 )$$

where δs is an adjustment to the current stimulus intensity parameter s.

A target ECAP amplitude is input to the feedback controller 310 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

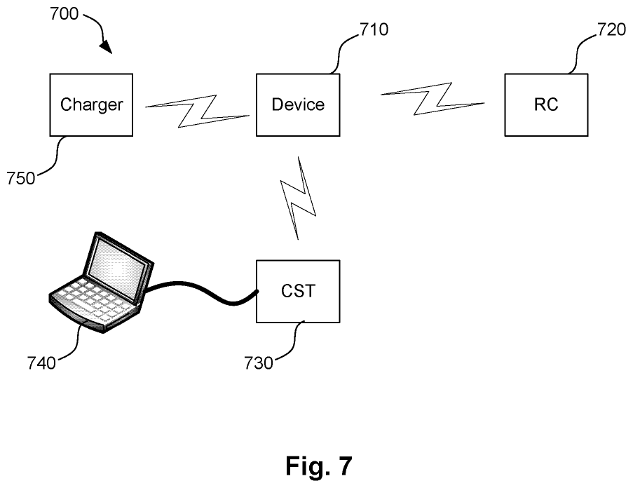

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

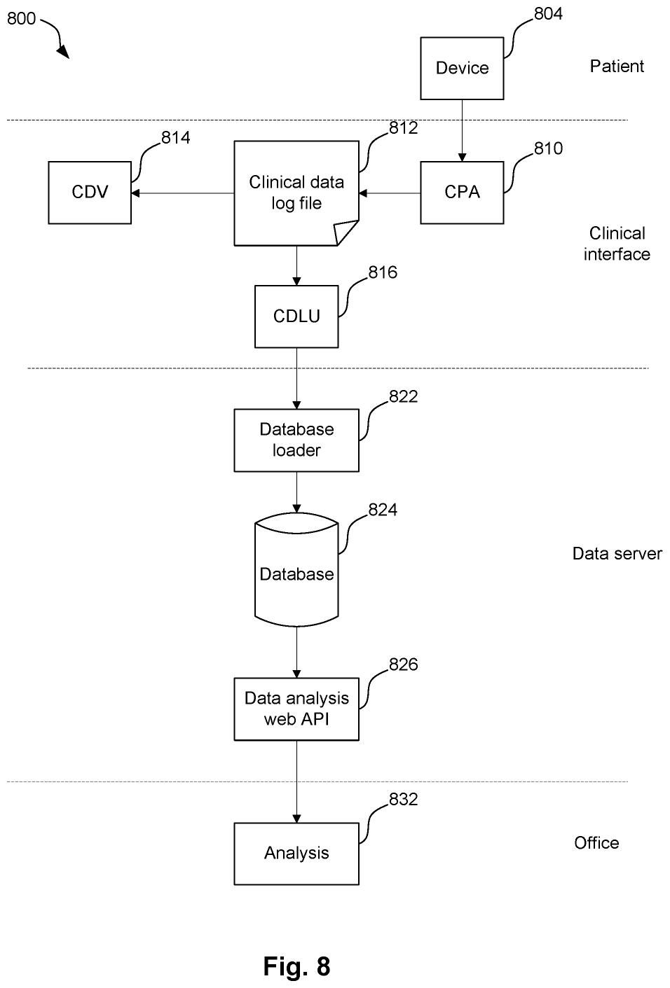

FIG. 8 is a block diagram illustrating the data flow 800 of a neural stimulation therapy system such as the system 700 of FIG. 7 according to one implementation of the present technology. Neuromodulation device 804, once implanted within a patient, applies stimuli over a potentially long period such as weeks or months and records neural responses, clinical settings, paraesthesia target level, and other operational parameters, discussed further below. Neuromodulation device 804 may comprise a Closed-Loop Neural Stimulation (CLNS) device, in that the recorded neural responses are used in a feedback arrangement to control clinical settings on a continuous or ongoing basis. To effect suitable SCS therapy, neuromodulation device 804 may deliver tens, hundreds or even thousands of stimuli per second, for many hours each day. The feedback loop may operate for most or all of this time, by obtaining sensed signals subsequent to every stimulus, or at least obtaining such sensed signals regularly. Each sensed signal generates a feedback variable such as a measure of the amplitude of the evoked neural response, which in turn results in the feedback loop changing at least one stimulus parameter for a following stimulus. Neuromodulation device 804 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 804 transmits data, e.g. via telemetry module 114, to a clinical programming application (CPA) 810 installed on a clinical interface. In one implementation, the clinical interface is the CI 740 of FIG. 7. The data can be grouped into two main sources: (1) Data collected in real-time during a programming session; (2) Data downloaded from a stimulator after a period of non-clinical use by a patient. CPA 810 collects and compiles the data into a clinical data log file 812.

All clinical data transmitted by the neuromodulation device 804 may be compressed by use of a suitable data compression technique before transmission by telemetry module 114 and/or before storage into the memory 118 to enable storage by neuromodulation device 804 of higher resolution data. This higher resolution allows neuromodulation device 804 to provide more data for post-analysis and more detailed data mining for events during use. Alternatively, compression enables faster transmission of standard-resolution clinical data.

The clinical data log file 812 is manipulated, analysed, and efficiently presented by a clinical data viewer (CDV) 814 for field diagnosis by a clinician, field clinical engineer (FCE) or the like. CDV 814 is a software application installed on the Clinical Interface (CI). In one implementation, CDV 814 opens one Clinical Data Log file 812 at a time. CDV 814 is intended to be used in the field to diagnose patient issues and optimise therapy for the patient. CDV 814 may be configured to provide the user or clinician with a summary of neuromodulation device usage, therapy output, and errors, in a simple single-view page immediately after log files are compiled upon device connection.

Clinical Data Uploader 816 is an application that runs in the background on the CI, that uploads files generated by the CPA 810, such as the clinical data log file 812, to a data server. Database Loader 822 is a service which runs on the data server and monitors the patient data folder for new files. When Clinical Data Log files are uploaded by Clinical Data Uploader 816, database loader 822 extracts the data from the file and loads the extracted data to Database 824.

The data server further contains a data analysis web API 826 which provides data for third-party analysis such as by the analysis module 832, located remotely from the data server. The ability to obtain, store, download and analyse large amounts of neuromodulation data means that the present technology can: improve patient outcomes in difficult conditions; enable faster, more cost effective and more accurate troubleshooting and patient status; and enable the gathering of statistics across patient populations for later analysis, with a view to diagnosing aetiologies and predicting patient outcomes.

### The Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient. Implementations of an Assisted Programming System (APS) according to the present technology are generally configured to meet this need.

In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient is analysed by the APM to determine the clinical settings for the neural stimulation therapy to be delivered by the stimulator 100. The APF is configured to complement the operation of the APM by responding to commands issued by the APM via the CST 730 to the stimulator 100 to deliver specified stimuli to the patient, and by returning, via the CST 730, measurements of neural responses to the delivered stimuli.

In other implementations, all the processing of the APS according to the present technology is done by the APF. In other words, the data obtained from the patient is not passed to the APM, but is analysed by the controller 116 of the device 710, configured by the APF, to determine the clinical settings for the neural stimulation therapy to be delivered by the stimulator 100.

In implementations of the APS in which the APM analyses the data from the patient, the APS instructs the device 710 to capture and return signal windows to the CI 740 via the CST 730. In such implementations, the device 710 captures the signal windows using the measurement circuit 128 and bypasses the ECAP detector 320, storing the data representing the raw signal windows temporarily in memory 118 before transmitting the data representing the captured signal windows to the APS for analysis.

Following the programming, the APS may load the determined program onto the device 710 to govern subsequent neural stimulation therapy. In one implementation, the program comprises clinical settings 121, also referred to as therapy parameters, that are input to the neuromodulation device 710 by, or stored in, the clinical settings controller 302. The patient may subsequently control the device 710 to deliver the therapy according to the determined program using the remote controller 720 as described above. The determined program may also, or alternatively, be loaded into the CPA for validation and modification.

### Measurement of Neural Response Characteristics

As mentioned above, according to one implementation of the ECAP detector, the sensed signals may be processed to determine the peak-to-peak ECAP amplitude in accordance with the teachings of International Patent Publication No. WO2015/074121. International Patent Publication No. WO2015/074121 discloses processing the sensed signal using a correlation detector, in which the sensed signal is correlated with a predetermined template referred to as the four-lobe filter. The value of the correlation, which may be positive, negative, or zero, is a measure both of the RMS magnitude of the neural response and of the extent to which that neural response is in phase with the template. The sign of the correlation is representative of the phase alignment between the neural response and the template; a positive sign indicates the two signals are in phase while a negative sign indicates the two signals are in antiphase. A correlation value of zero indicates the two signals are orthogonal, i.e. in quadrature, or ninety degrees out of phase. The phase alignment is dependent on the offset at which the correlation is calculated, so International Patent Publication No. WO2015/074121 also discloses a method of choosing an offset that optimally aligns the template with the sensed signal, so that the correlation value reflects the RMS magnitude of any neural response in the sensed signal. This process is effective provided the template morphologically resembles the neural response. The correlation value so calculated may be scaled by a predetermined scalar to convert the RMS magnitude to a measure of peak-to-peak amplitude of the neural response.

In addition to being chosen to resemble a neural response, the template is chosen to be partially orthogonal to an artefact component in the sensed signal, whereby the artefact component is modelled as the sum of two decaying exponentials with different time constants. The correlation measure is therefore somewhat insensitive to artefact satisfying such a model in the sensed signal.

However, due to the lack of perfect orthogonality, some artefact component will “leak through” into the correlation value. In addition, this artefact model may be incomplete in that certain artefacts are not well modelled as the sum of two decaying exponentials with different time constants, regardless of what time constants are chosen. The template will be even less orthogonal to such artefacts, with the result that further artefact component will “leak through” into the correlation value. In addition, the template, no matter how carefully the delay is chosen, will not be exactly in phase with the neural response component of the sensed signal. Both of these effects lessen the accuracy of the correlation value as a measure of magnitude of the neural response component. The present disclosure derives a template for a correlation-based ECAP detector that is less prone to at least one of these effects. Such a template would provide improved accuracy of neural response measurement under a wider variety of artefact conditions, or at least provide an alternative.

In the following, the term “artefact” may be taken to mean “any undesired, non-stochastic signal in the captured signal window”. One example is stimulus artefact, as described above. However, the term includes other non-ECAP-related neural responses such as EMGs or late responses.

The sensed signal may be written as a vector y of samples in the captured signal window. A correlation detector may be implemented as an inner product (dot product) of the sensed signal y and a template vector d, returning a correlation value C:

$$C = 〈 y , d 〉 \qquad ( 4 )$$

The template d is normalised so that it has unit norm:

$$‖ d ‖ = 1 \qquad ( 5 )$$

where the norm of a vector is its RMS magnitude.

The sensed signal y may be modelled as a sum of a neural response component y_{E}, an artefact component y_{A}, and a noise signal e:

$$y = {y}_{E} + {y}_{A} + e \qquad ( 6 )$$

Since correlation is linear, the correlation in Equation (4) may be expanded as

$$C = 〈 {y}_{E} , d 〉 + 〈 {y}_{A} , d 〉 + 〈 e , d 〉 \qquad ( 7 )$$

The aim of the detector is to measure the RMS magnitude (norm) of the neural response component y_{E}. However it may be seen that the presence of an artefact component y_{A} means that in general, even if the template d is a normalised replica of the neural response component y_{E} (a matched filter), the correlation value C is not equal to ∥y_{E}∥.

If the noise vector is identically independently distributed (IID) Gaussian noise of zero mean, then the inner product of template d and noise e will also be IID Gaussian, of zero mean, with the same variance as the noise vector e. That is, if the variance Var[e] of the noise vector e is σ², the variance of the inner product is also σ², as shown by the following series of identities:

$$Var ( \sum d [ i ] e [ i ] ) = \sum {d [ i ]}^{2} Var ( e [ i ] ) = {σ}^{2} \sum {d [ i ]}^{2} = {σ}^{2} \qquad ( 8 )$$

The artefact component y_{A} can be reliably modelled as an arbitrary linear combination of r basis functions φᵢ. That is, for any artefact component y_{A}, a set of coefficients {aᵢ: i=1, . . . , r} may be found such that the artefact component is the weighted sum of the basis functions φᵢ, with weights aᵢ:

$${y}_{A} = {\sum }_{i = 1}^{r} {a}_{i} {ϕ}_{i} \qquad ( 9 )$$

The artefact basis [symbol] ={φᵢ, i=1, . . . , r} may be assumed to be orthonormal. (If it is not orthonormal, an orthonormalization such as Gramm-Schmidt may be applied to the basis to render it orthonormal.) The coefficients aᵢ that model the artefact component y_{A} may therefore be found by inner products of the artefact component y_{A} with the basis functions φᵢ:

$${a}_{i} = 〈 {y}_{A} , {ϕ}_{i} 〉 \qquad ( 10 )$$

The optimal template d maximises the value of [symbol] y_{E}, d [symbol] with respect to [symbol] e, d [symbol] (the signal-to-noise ratio of the correlation detector) while ensuring [symbol] y_{A}, d [symbol] is zero for any artefact component y_{A}, i.e. the template is orthogonal to the artefact basis [symbol] . Using the expansion in Equation (9), orthogonality to the artefact basis may be ensured if d is orthogonal to all the artefact basis functions φ:

$$〈 d , {ϕ}_{i} 〉 = 0 for all i = 1 , … , r \qquad ( 11 )$$

The template d that simply maximises the signal-to-noise ratio of the correlation detector is

$$\frac{{y}_{E}}{‖ {y}_{E} ‖} ,$$

i.e. a normalised matched filter. If such a template d satisfied Equation (11), the correlation value C would simply be the RMS magnitude of the neural response component y_{E}, which is the desired output of the correlation detector.

However, it is rarely the case that [symbol] y_{E}, φᵢ [symbol] =0, i.e. pure neural responses are rarely orthogonal to artefact. A template d other than a matched filter is therefore advantageous.

Because the artefact basis [symbol] is orthonormal, the least-squares approximation to a sensed signal y by the artefact basis [symbol] may be found by computing inner products of the sensed signal y with the basis functions φᵢ:

$${\hat{a}}_{i} = 〈 y , {ϕ}_{i} 〉 \qquad ( 12 )$$

The ordinary-least-squares (OLS) coefficients âᵢ represent the projection of the sensed signal y onto the artefact basis [symbol] . If an un-normalised template \hat{d} is defined as the residual of the projection of the sensed signal y onto the artefact basis [symbol] , i.e.

$$\hat{d} = y - {\sum }_{i = 1}^{r} {\hat{a}}_{i} {ϕ}_{i} \qquad ( 13 )$$

it may be shown that the un-normalised template \hat{d} is orthogonal to the artefact basis [symbol] , i.e. \hat{d} satisfies Equation (11).

It follows that the correlation of the un-normalised template \hat{d} with an arbitrary artefact-containing sensed signal y′ is simply the correlation of the neural response component y_{E}′ of the sensed signal y′ with \hat{d} (neglecting noise). In other words, the un-normalised template \hat{d} is wholly insensitive to artefact as represented by the artefact basis [symbol] :

$$〈 \hat{d} , {y}^{′} 〉 = 〈 \hat{d} , {y}_{E}^{′} 〉 \qquad ( 14 )$$

It may also be shown that a normalised version d₀ of the un-normalised template \hat{d} maximises the signal-to-noise ratio of the correlation detector while still remaining wholly insensitive to artefact. The template d₀ according to the present technology is referred to as the Bespoke Unitary Residual Detector (BURD).

Conveniently in SCS, the morphology of an ECAP is approximately constant with stimulus intensity. That is,

$${y}_{E} = α ( s ) E \qquad ( 15 )$$

where s is the stimulus intensity parameter, E is a normalised ECAP template and α(s) is the stimulus-intensity-dependent intensity of the ECAP component y_{E}. So, for all sensed signals y obtained subsequent to stimulation at an arbitrary stimulus intensity parameter s, the correlation value C returned by a correlation detector using the BURD d₀ may be shown to be given by

$$C = α ( s ) {C}_{E} \qquad ( 16 )$$

where C_{E} is a constant correlation factor equal to <d₀, E>. The correlation detector output's variation with stimulus intensity is therefore a scaled version of the evoked neural response's intensity variation with stimulus intensity. This is a beneficial property of an effective feedback variable. The signal-to-noise ratio of such a correlation detector increases to the extent that d₀ resembles the ECAP template E, so it makes sense to derive the BURD d₀ from sensed signals that are known to contain ECAPs.

A template for a correlation detector according to the present technology may therefore be derived by applying Equation (13) to a representative sensed signal y_{R} containing a non-zero ECAP component y_{E} and normalising the resulting template d to obtain the BURD d₀. In one implementation, the representative signal y_{R} may be derived by averaging or accumulating multiple sensed signals that are known to contain ECAPs.

An ECAP discriminator may be applied to a sensed signal y to determine whether or not the sensed signal contains an ECAP. In one implementation, such an ECAP discriminator is the Noise Departure Detector (NDD).

### Noise Departure Detector (NDD)

The NDD is a statistical detector of the presence of an ECAP in a signal window. The operation of the NDD on a signal window may be preceded by an “artefact scrubber” which removes artefact from the signal window. On such artefact scrubber is disclosed in International Patent Publication no. WO2020/124135, the entire contents of which are herein incorporated by reference. The NDD works by detecting a statistically unusual difference from the expected noise present in a signal window, which difference is attributed to the presence of an ECAP in the signal window.

The calibration of an NDD may be carried out on one or more captured signal windows which are known not to contain evoked neural responses. In one implementation, such signal windows are “zero current” signal windows which are captured when no stimulus has been applied, and which have been scrubbed for artefact, and may therefore be treated as comprising only noise. The calibration comprises forming estimates of parameters of a predetermined “noise model” (statistical distribution) from the samples in the one or more “zero current” signal windows. In one implementation, the noise model is Gaussian and the parameters are the mean \tilde{μ} and standard deviation \hat{σ} of the samples.

Once calibrated, an NDD may be applied to a signal window by counting the number \tilde{k} of outliers in the signal window, i.e. the number of samples in the signal window that depart significantly from the noise model. For a Gaussian noise model, the NDD counts the number \tilde{k} of samples that differ from the mean estimate \tilde{μ} by more than n times the standard deviation estimate \hat{σ}, where n is a small integer. The number \tilde{k} of outliers is compared to the number \hat{k} of samples that would be expected to occur if the signal window consisted solely of noise with mean \tilde{μ} and standard deviation \hat{σ}. The difference between \tilde{k} and \hat{k} is divided by the number of samples N in the signal window to obtain a metric r that quantifies the ratio of outliers present in a signal window relative to the expected ratio of outliers in a signal window that obeys the noise model.

It may be shown that for Gaussian noise model, the NDD may estimate the metric r as

$$r = \frac{\tilde{k}}{N} - 2 Φ ( \frac{n}{2} ) \qquad ( 17 )$$

where Φ is the standard normal cumulative distribution function.

A negative or zero value of the metric r indicates a signal window consistent with the noise model, whereas a positive value of r indicates a departure from the noise model. Such a departure is deemed to be due to the presence of an ECAP in the signal window.

In one implementation of the NDD, n is set to 3. Smaller values of n make the NDD more sensitive, indicating a departure from noise more readily and increasing the rate of Type I errors (false positives). Conversely, high values for n necessitate large outliers before r will indicate a noise departure, increasing the rate of Type II errors (false negatives).

In one implementation of the NDD, a sigmoid function may be applied to the raw metric r to map the metric r to a quality indicator Q_{NDD} in the interval [0, 1]:

$${Q}_{NDD} = \frac{1}{1 + exp ( - γ r )} \qquad ( 18 )$$

where γ is a parameter that balances the Type I and Type II errors. The quality indicator Q_{NDD} has a natural interpretation: Q_{NDD}<0.5 corresponds to r≤0 and indicates that the signal window is most likely noise. Conversely, Q_{NDD}>0.5 indicates a departure from the noise model that is deemed to represent an ECAP. In one implementation, γ is set to 50.

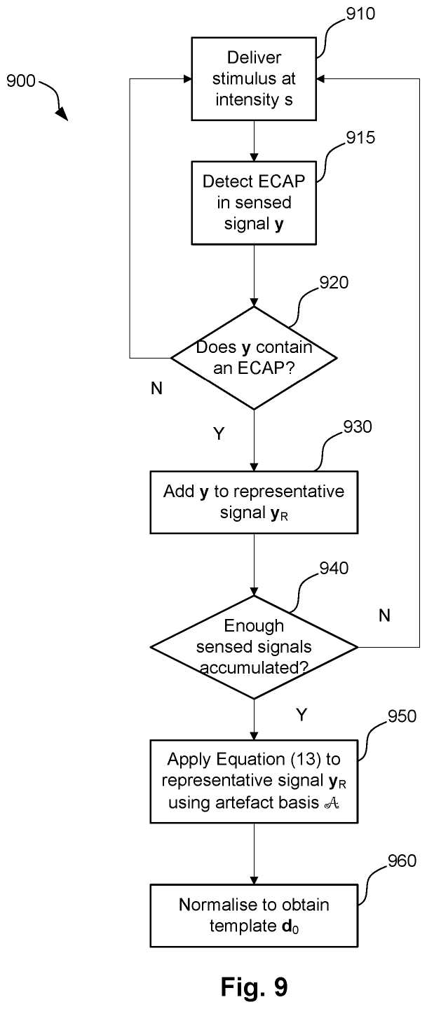

FIG. 9 is a flow chart illustrating a method 900 of deriving a template d₀ (a BURD) for a correlation-based ECAP detector such as the ECAP detector 320 forming part of the CLNS system 300. The method 900 may be carried out by the CI 740 as configured by the APM, by the controller of the device 710 as configured by the APF, or by some combination of the two working in concert as described above. The output of the method 900, a template d₀ for a correlation-based ECAP detector, may be stored as part of the therapy parameters that are input to the neuromodulation device 710 by the Assisted Programming System.

The method 900 starts at step 910, which delivers a stimulus at a fixed stimulus intensity s. Step 915 then applies an ECAP discriminator such as the NDD to a sensed signal y in a captured signal window to detect whether an ECAP is present in the sensed signal y. Step 920 then tests the output of the ECAP discriminator. If an ECAP was not present in the sensed signal y (“N”), the method 900 returns to step 910. If an ECAP was present in the sensed signal y (“Y”), step 930 adds the sensed signal y to a representative signal y_{R} (which was initialised at zero before starting the method 900). Optionally, before executing step 930, some integrity checking may be carried out on the sensed signal y. If the sensed signal y fails the integrity checking, step 930 is bypassed. One example of integrity checking is to check whether any samples in the sensed signal y are clipped, i.e. have values at either extreme of the output range of the measurement circuitry 318.

Step 940 then tests whether enough sensed signals containing ECAPs have been accumulated to the representative signal y_{R}. If not (“N”), the method 900 returns to step 910. If so (“Y”), step 950 applies Equation (13) to the representative signal y_{R} using an artefact basis [symbol] to obtain the un-normalised template \hat{d}. Optionally, step 950 may first divide the representative signal y_{R} by the number of sensed signals accumulated into the representative signal y_{R}, thereby making the representative signal y_{R} into the average of all the sensed signals. Finally, step 960 normalises the un-normalised template \hat{d} to obtain the BURD d₀ to be used by the correlation-based ECAP detector as part of the therapy program.

### Artefact Bases

As mentioned above, the derivation of the BURD d₀ requires an artefact basis [symbol] . The effectiveness of the BURD d₀ in producing feedback variables for an ECAP-driven CLNS system improves according to the effectiveness of the artefact basis [symbol] in representing artefact components of sensed signals in all their variegated complexity.

In one implementation, in which the artefact is stimulus artefact, the artefact basis [symbol] is derived from a constant phase element (CPE) model of the electrode-tissue interface. This CPE basis is described in the above-mentioned International Patent Publication no. WO2020/124135. The CPE basis [symbol] _{CPE} comprises five basis functions φᵢ (where i=1, . . . , 5) that are defined for a biphasic stimulus waveform as:

$${ϕ}_{1} ( t ) = s ( t ) - s ( t - pw ) - s ( t - pw - ipg ) + s ( t - 2. pw - ipg ) \qquad ( 19 )$$

$${ϕ}_{2} ( t ) = i ( t ) - i ( t - pw ) - i ( t - pw - ipg ) + i ( t - 2 . pw - ipg ) \qquad ( 20 )$$

$${ϕ}_{3} ( t ) = s ( t - pw - ipg ) - s ( t - 2 . pw - ipg ) \qquad ( 21 )$$

$${ϕ}_{4} ( t ) = i ( t - pw - ipg ) - i ( t - 2 . pw - ipg ) \qquad ( 22 )$$

$${ϕ}_{5} ( t ) = 1 \qquad ( 23 )$$

where pw is the pulse width of each phase of the biphasic stimulus, ipg is the inter-phase gap between the two phases, i(t) is the impulse response of a CPE, defined as a decaying exponential:

$$i ( t ) = {t}^{- α} , t \geq 0 \qquad ( 24 )$$

and s(t) is the step response of a CPE, defined as the integral of the impulse response i(t) over all positive time t:

$$s ( t ) = {\int }_{t = 0}^{t = ∞} {t}^{- α} dt = \frac{1}{1 - α} {t}^{1 - α} + 𝒞 \qquad ( 25 )$$

The parameter α is a constant that depends on the geometry of the electrode-tissue interface, but in one implementation may be set to 0.364.

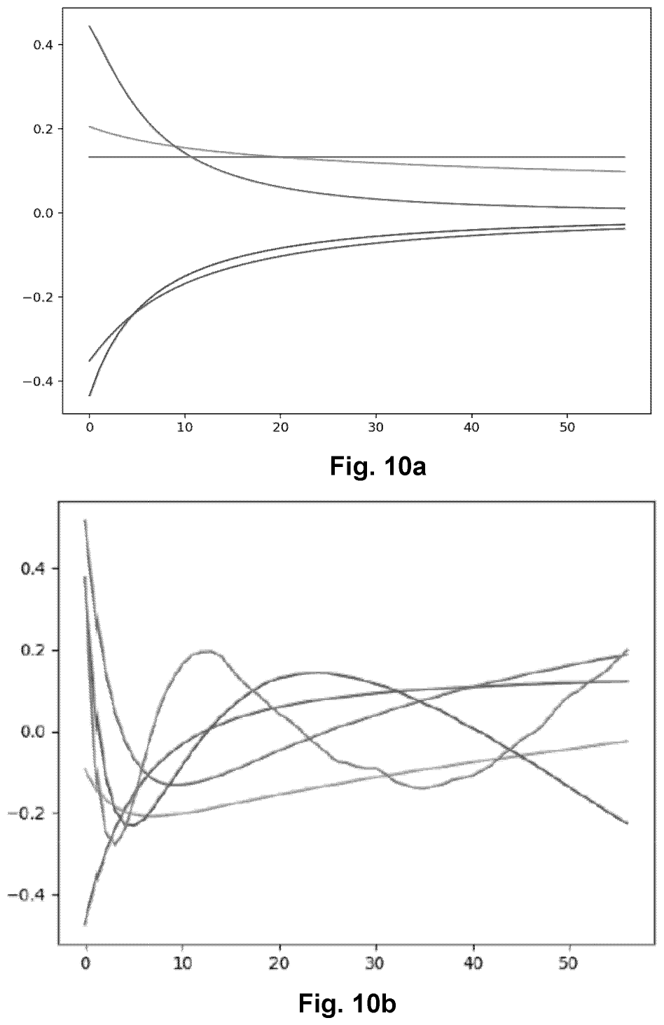

FIG. 10a contains a graph showing the five basis functions φᵢ in the CPE artefact basis [symbol] _{CPE}.

Applying Equation (13) to a representative signal y_{R}, as at step 950 of the method 900, requires the OLS coefficients âᵢ of the artefact component of the representative signal y_{R} in the artefact basis [symbol] . The CPE basis [symbol] _{CPE} is not orthonormal, so the OLS coefficients âᵢ may not be found simply by applying Equation (12) to the representative signal y_{R}. However, the coefficients âᵢ may be found by orthonormalizing the CPE basis [symbol] _{CPE}, applying Equation (12) to the orthonormalized version of the CPE basis [symbol] _{CPE}, and then transforming the resulting coefficients back to the original CPE basis [symbol] _{CPE} using conventional linear algebra.

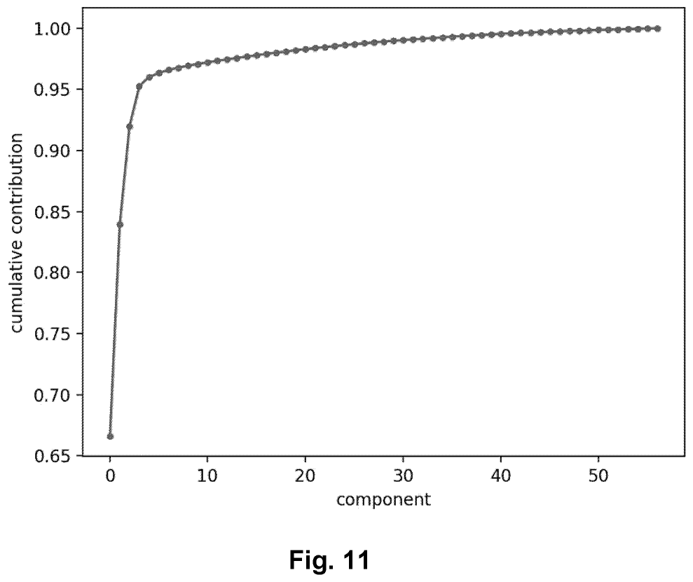

In an alternative implementation, an artefact basis [symbol] _{SVD} suitable for any form of artefact may be obtained from the Singular Value Decomposition (SVD) of a library X of sensed signals y_{NR} containing no neural response component, only artefact components and noise. The SVD is a machine learning technique for obtaining a basis that optimally represents a given training data set. The library X may be constructed as a matrix whose columns are the no-response sensed signals y_{NR}. In one implementation, such a library X may be obtained from a database of sensed signals from different patients, such as the database 824. The library X may be obtained by extracting from the database sensed signals y captured subsequent to stimuli of stimulus intensity s less than half the ECAP threshold, with the same pulse width and inter-phase gap. The SVD decomposes the matrix X into a product of three matrices U, Σ, and V*. The r basis functions φᵢ in the SVD artefact basis [symbol] _{SVD}, which are by definition orthonormal, may be obtained as the first r columns of the SVD matrix U corresponding to the largest r singular values of the matrix X. FIG. 10b contains a graph showing the first five (i.e. r=5) basis functions φᵢ in an SVD artefact basis [symbol] _{SVD} obtained in this way from a library X of no-response sensed signals y_{NR}. These first five basis functions φᵢ provide an adequate approximation to the artefact-only sensed signals in the library X, in the sense that they explain 96% of the power in the matrix X. FIG. 11 contains a graph showing the explanatory power of the SVD artefact basis [symbol] _{SVD} obtained from the matrix X as a function of the rank r of [symbol] _{SVD}.

The SVD artefact basis [symbol] _{SVD} may be personalised to a specific patient by deriving it from a library X of no-response sensed signals y_{NR} specific to that patient. This will in general lead to an artefact basis of lower rank r with the same explanatory power than would be obtained from a library X of no-response sensed signals from a population comprising multiple patients. Since the SNR of a correlation detector derived using Equation (13) tends to decrease as the rank r of the artefact basis increases, a patient-specific SVD artefact basis [symbol] _{SVD} may be preferable to a population-based SVD artefact basis.

Furthermore, a patient-specific SVD artefact basis [symbol] _{SVD} may be adapted over time by repeatedly recomputing it from anew library X of no-response sensed signals captured during therapy of the patient.

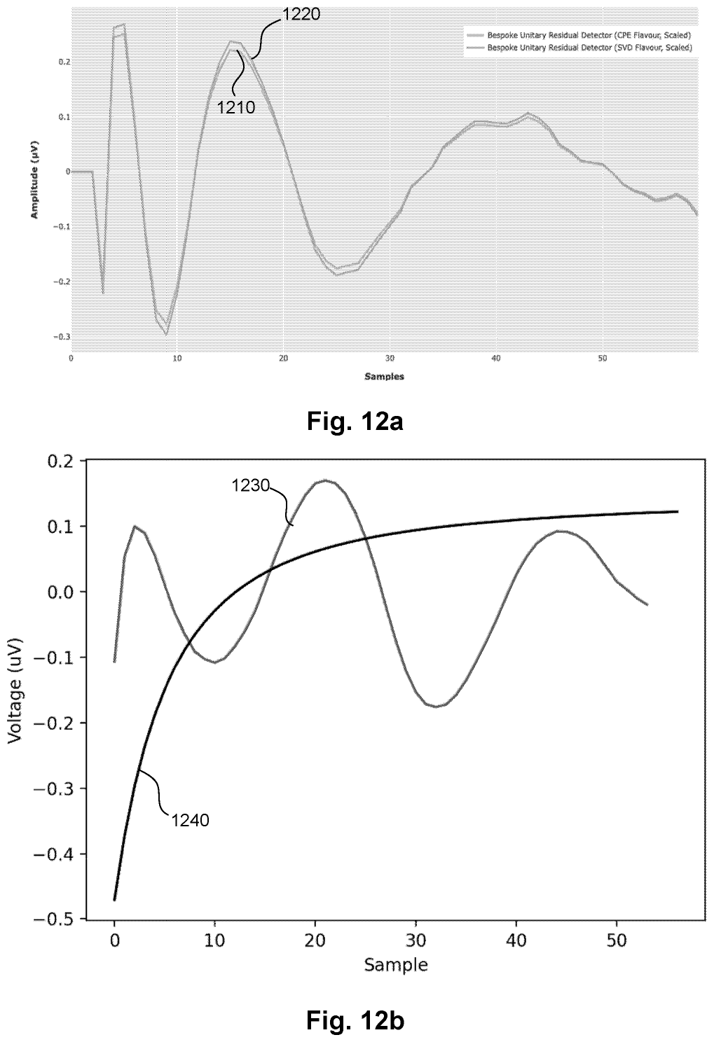

FIG. 12a contains a graph containing a trace 1210 representing the BURD d₀ obtained from the above-described CPE artefact basis [symbol] _{CPE}. The graph also contains a trace 1220 representing the BURD d₀ obtained from the above-described SVD artefact basis [symbol] _{SVD}. It may be seen that the two BURDs closely resemble each other.

In some implementations, instead of applying Equation (13) to a representative signal y_{R}, as at step 950 of the method 900, to obtain the BURD d₀, the BURD d₀ may be derived by applying Equation (13) to the four-lobe filter d_{FL} described in International Patent Publication No. WO2015/074121. The resulting BURD d₀ is a warped version of the four-lobe filter d_{FL}. Such implementations are advantageous because the four-lobe filter d_{FL} is known to be effective as a correlation template for measuring an ECAP characteristic in sensed signals, and is therefore plausible as representative signal y_{R} containing an ECAP component. FIG. 12b contains a graph containing a trace 1230 representing the BURD d₀ obtained from the four-lobe filter d_{FL}. It may be seen that the BURD trace 1230 is not dissimilar in morphology to the two BURD traces 1210 and 1220 graphed in FIG. 12a. FIG. 12b also contains a trace 1240 representing a typical artefact component, for comparison.

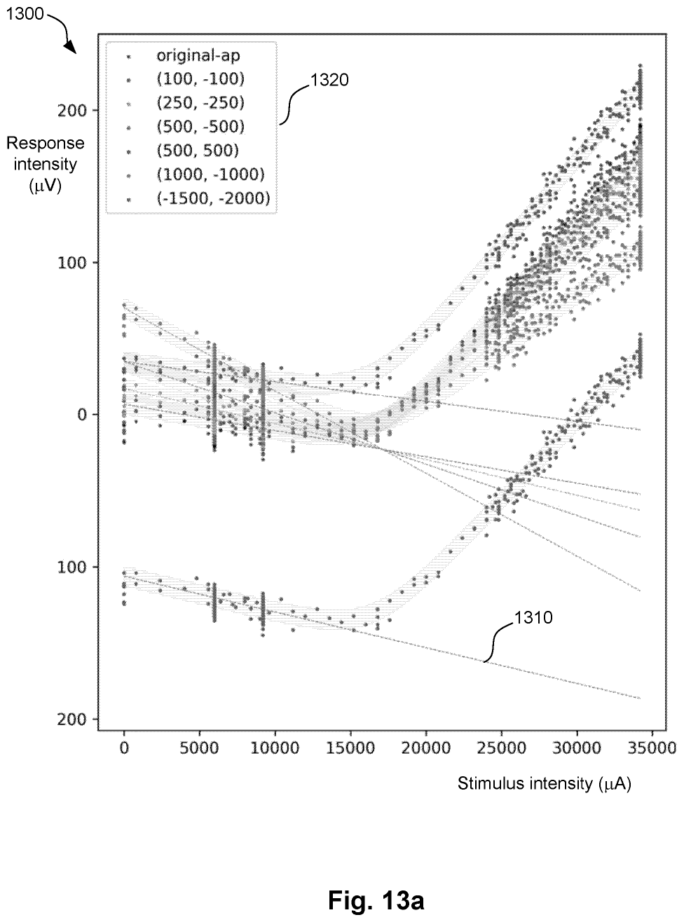

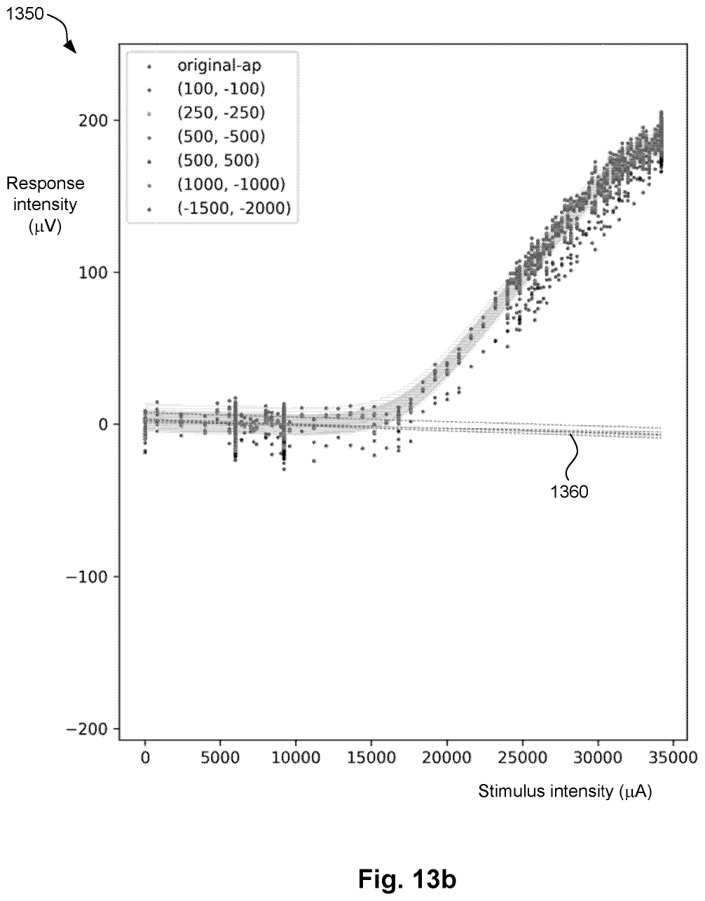

FIG. 13a contains a graph 1300 containing response intensities estimated using the four-lobe filter plotted against stimulus intensity. The response intensities were obtained by applying the four-lobe filter to simulated signal windows obtained using different models of the artefact dependence on stimulus intensity. Each artefact model is linear and is parametrised by a (slope, intercept) pair of parameters as indicated by the legend 1320. To obtain the simulated signal windows for each artefact model, simulated artefact components obtained by scaling a representative artefact according to the model and the corresponding stimulus intensity were added to a set of raw signal windows captured at the respective stimulus intensities. The leakage of artefact into the estimated response intensities at sub-threshold stimulus intensities may be clearly seen from the divergence of the response intensities towards the left side of the graph 1300 from the ideal, horizontal form of the activation plot in the sub-threshold region, as illustrated in FIG. 4a. A perfectly artefact-insensitive template would return identical response intensities at each stimulus intensity, independent of the magnitude of the added artefact. The response intensities in the graph 1300 do not have this property, being widely divergent at any given stimulus intensity. Each dashed line, e.g. the line 1310, represents an estimate of the artefact component contribution to the response intensity as modelled by a linear dependence on stimulus intensity in the sub-threshold region. The divergence of the dashed lines shows the significant artefact components leaking through the four-lobe filter.

FIG. 13b contains a graph 1350 containing response intensities estimated using a BURD template d₀ derived from an SVD artefact basis, plotted against stimulus intensity. The response intensities were obtained by applying the BURD template d₀ to the same simulated signal windows as were used to obtain FIG. 13a. The SVD artefact basis was itself derived from the simulated artefact components used to produce the simulated signal windows as described above. The much greater similarity of the response intensities in the graph 1350 to each other at each stimulus intensity, compared to those in the graph 1300, demonstrates the greater robustness of the BURD template d₀ to artefact compared to the four-lobe filter. Each dashed line, e.g. the line 1360, as in FIG. 13a, represents an estimate of the artefact component contribution to the response intensity as modelled by a linear dependence on stimulus intensity in the sub-threshold region. The similarity of these dashed lines to each other further illustrates the robustness of the BURD template d₀ to artefact.

### Quantising the BURD Template

In some implementations of the BURD template d₀ within the ECAP detector 320, a fixed-point representation of the BURD template d₀ may be used. To prepare for such implementations, the coefficients of the BURD template d₀, once derived according to the method 900, may be quantised to a fixed number of bits (e.g. 14 bits) before being stored as part of the therapy parameters.

As mentioned above in Equation (23), the fifth basis function φ₅(t) of the CPE basis [symbol] _{CPE} is a constant function (φ₅(t)=1). This means that BURD template d₀, if derived using the CPE basis [symbol] _{CPE}, must be orthogonal to any constant value. In other words, the coefficients of the BURD template d₀ must sum to zero. In order to preserve this property after quantisation to a fixed-point representation, a “walkback” algorithm may be employed. The aim of the walkback algorithm is to distribute any necessary adjustment to the quantised coefficients among the coefficients, starting at the endmost coefficient and working backward until the cumulative adjustment is sufficient to make the quantised coefficients sum to zero.

The walkback algorithm operates as follows.
1. A delta value is calculated as the sum of the template coefficients. If the delta value is zero, the template already rejects a constant and there is no need to proceed further.
2. A walkback index is set to the position of the last coefficient in the template.
3. The delta value is subtracted from the coefficient pointed to by the walkback index, to the extent that the result does not exceed the fixed-point range of the coefficient, i.e. to the extent that the subtraction does not take the coefficient outside the range [−2ⁿ⁻¹, 2ⁿ⁻¹−1] where n is the bit depth of the fixed-point representation.
4. The walkback index is decremented by 1.
5. The delta value is updated to the recalculated sum of the template coefficients.
6. The algorithm repeats from step 3 until the delta value reaches zero.

### Maintaining an Effective BURD Template

The derivation of the BURD template d₀ according to the method 900 is dependent on the measured ECAP and its morphology. The ECAP morphology in turn is dependent on several therapy parameters. These BURD-related therapy parameters include:
Stimulus electrode configuration
Measurement electrode configuration
The ratio of current on each stimulus electrode relative to other stimulus electrodes
Pulse width
Interphase gap
Pulse shape (triphasic, biphasic) and polarity (positive first, negative first)
Sampling delay
Sampling period
Inter-stimulus interval
Shorting interval
Number of stimulation sets in a multiple-stimulation-set program
Position of the applied stimulation set (from which ECAP measurements are made) in a multiple-stimulation-set program

Modifying any of these therapy parameters could cause the ECAP morphology to change and in turn, de-tune the BURD template, thereby rendering ECAP characteristic measurements returned by the BURD template inaccurate.

New therapy parameters may be received when a new therapy program is sent to the stimulator 100 (prior to starting therapy) or via program updates (once therapy has commenced).

According to an aspect of the present technology, a number representative of the relevant current therapy parameters may be calculated and compared with the number calculated in the same way from the relevant therapy parameters of any new or modified program. Based on the comparison, the BURD template is re-derived, for example according to the method 900 of FIG. 9. This allows an effective BURD template to be maintained as therapy parameters change.

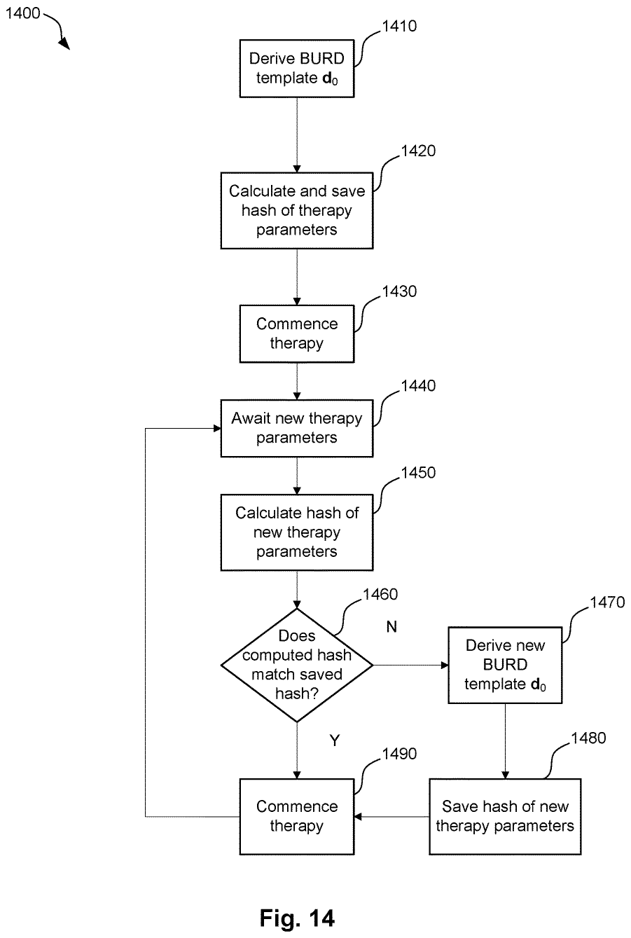

FIG. 14 is a flow chart illustrating a method 1400 of maintaining an effective BURD template as therapy parameters change. The method 1400 may be carried out by the CI 740 as configured by the APM, by the controller of the device 710 as configured by the APF, or by some combination of the two working in concert as described above. The method 1400 is triggered when the therapy parameters of a therapy program are determined at the end of a programming session.

The method 1400 starts at step 1410, which derives a BURD template from the therapy parameters, for example using the method 900 described above. Step 1410 is optional, and may be omitted if the BURD template already forms part of the therapy parameters as described above. Step 1420 then calculates and saves, for example to the memory 118 of the stimulator 100, a hash of the values of the therapy parameters on which the BURD template's effectiveness is dependent, for example the BURD-related therapy parameters listed above. In one implementation, step 1420 uses a 16-bit Cyclic Redundancy Check (CRC) to calculate the hash of the BURD-related therapy parameters.

At the next step 1430, CLNS therapy using the therapy parameters commences as described above.

Step 1440 follows, which awaits new therapy parameters (or an update to the existing therapy parameters). Once new therapy parameters are received, step 1450 calculates a hash of the values of the new BURD-related therapy parameters among the new therapy parameters using the same method as was used in step 1420. Step 1460 then compares the computed hash with the saved hash to determine if the hashes match. If the hashes do not match (“N”), step 1470 derives a new BURD template, for example using the method 900, using the new therapy parameters. Step 1480 then saves the hash of the new therapy parameters, and at step 1490, CLNS therapy using the new therapy parameters commences as in step 1430. The method 1400 then returns to step 1440. If the hashes do match (“Y”), the method 1400 proceeds directly to step 1490.

In an alternative implementation, the hash is calculated as part of the programming session and becomes part of the therapy parameters. In such an implementation, steps 1420 and step 1450 are omitted. Instead, the hashes are simply retrieved from the therapy parameters in order to be compared at step 1460.

The advantage of the method 1400 in relation to a comparison of the therapy parameter values themselves is that it is more efficient to compare hashes than to individually compare the values of each of a list of BURD-related therapy parameters. The hash is computed in such a way that a small change to any one of the BURD-related therapy parameter values produces a detectable change in the hash, even though the hash may be represented by many fewer bits than the therapy parameter values themselves. However, step 1460 may, after finding a match (“Y”), optionally compare the values of each of the BURD-related therapy parameters to confirm that there has been no change in the values of such parameters. If such confirmation is not forthcoming, the method 1400 may proceed to step 1470.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

LABEL LIST

| | | | |
|---|---|---|---|
| Stimulator | 100 | ECAP threshold | 512 |
| Patient | 108 | ECAP target | 520 |
| electronics module | 110 | ECAP | 600 |
| Battery | 112 | neural stimulation system | 700 |
| telemetry module | 114 | neuromodulation device | 710 |
| controller | 116 | remote controller | 720 |
| memory | 118 | CST | 730 |
| clinical data | 120 | CI | 740 |
| clinical settings | 121 | charger | 750 |
| control programs | 122 | data flow | 800 |
| pulse generator | 124 | neuromodulation device | 804 |
| electrode selection module | 126 | CPA | 810 |
| measurement circuitry | 128 | clinical data log file | 812 |
| ground | 130 | CDV | 814 |
| electrode array | 150 | clinical Data Uploader | 816 |
| biphasic stimulus pulse | 160 | database loader | 822 |
| neural response | 170 | database | 824 |
| nerve | 180 | data analysis web API | 826 |
| communications channel | 190 | analysis module | 832 |
| external device | 192 | method | 900 |
| system | 300 | step | 910 |
| clinical settings controller | 302 | step | 915 |
| target ECAP controller | 304 | step | 920 |
| box | 308 | step | 940 |
| box | 309 | step | 950 |
| feedback controller | 310 | step | 960 |
| box | 311 | BURD trace | 1210 |
| stimulator | 312 | BURD trace | 1220 |
| element | 313 | trace | 1230 |
| measurement circuitry | 318 | trace | 1240 |
| ECAP detector | 320 | graph | 1300 |
| comparator | 324 | line | 1310 |
| gain element | 336 | legend | 1320 |
| integrator | 338 | graph | 1350 |
| activation plot | 402 | line | 1360 |
| ECAP threshold | 404 | method | 1400 |
| discomfort threshold | 408 | step | 1410 |
| perception threshold | 410 | step | 1420 |
| therapeutic range | 412 | step | 1430 |
| activation plot | 502 | step | 1440 |
| activation plot | 504 | step | 1450 |
| activation plot | 506 | step | 1460 |
| ECAP threshold | 508 | step | 1470 |
| ECAP threshold | 510 | step | 1480 |
| step | 1490 | | |

1. An implantable device for controllably delivering neural stimuli, the implantable device comprising:
a plurality of electrodes including one or more stimulus electrodes and one or more measurement electrodes;
a stimulus source configured to provide neural stimuli to be delivered via the one or more stimulus electrodes to a neural pathway of a patient in order to evoke neural responses from the neural pathway;
measurement circuitry configured to capture signal windows from signals sensed on the neural pathway via the one or more measurement electrodes subsequent to respective neural stimuli; and
a control unit configured to:
control the stimulus source to provide a neural stimulus according to a stimulus intensity parameter; and
measure an intensity of an evoked neural response in a signal window captured subsequent to the neural stimulus by correlating the captured signal window with a template,
wherein the template is orthogonal to a predetermined artefact basis that models an artefact component in the captured signal window.

2. The implantable device of claim 1, wherein the control unit is further configured to:
determine a feedback variable from the measured intensity of the evoked neural response; and
adjust, using a feedback controller, the stimulus intensity parameter so as to maintain the feedback variable at a target value.

3. The implantable device of claim 1, wherein the control unit is further configured to derive the template based on a comparison between new therapy parameters and therapy parameters according to which the neural stimulus was provided.

4. The implantable device of claim 3, wherein the control unit is configured to derive the template by:
projecting a representative signal comprising an evoked neural response component onto the predetermined artefact basis; and
deriving the template by subtracting the projected representative signal from the representative signal.

5. The implantable device of claim 4, wherein the control unit is further configured to obtain the representative signal from one or more signal windows captured subsequent to respective stimuli delivered using the new therapy parameters.

6. An automated method of measuring an evoked response to neural stimuli delivered to a neural pathway of a patient, the method comprising:
delivering a neural stimulus to the neural pathway of the patient in order to evoke a neural response from the neural pathway, the neural stimulus being delivered according to a stimulus intensity parameter;
capturing a signal window sensed on the neural pathway subsequent to the delivered neural stimulus; and
measuring an intensity of a neural response evoked by the delivered neural stimulus in the captured signal window by correlating the captured signal window with a template,
wherein the template is orthogonal to a predetermined artefact basis that models an artefact component in the captured signal window.

7. The method of claim 6, further comprising:
determining, from the measured intensity of the evoked neural response, a feedback variable; and
adjusting, using the feedback variable, the stimulus intensity parameter so as to maintain the feedback variable at a target value.

8. The method of claim 7, further comprising deriving the template based on a comparison between new therapy parameters and therapy parameters according to which the neural stimulus was delivered.

9. The method of claim 8, wherein deriving the template comprises:
projecting a representative signal comprising an evoked neural response component onto the predetermined artefact basis; and
deriving the template by subtracting the projected representative signal from the representative signal.

10. The method of claim 9, further comprising obtaining the representative signal from one or more signal windows captured subsequent to respective stimuli delivered using the new therapy parameters.

11. A neural stimulation system comprising:
an implantable device for controllably delivering neural stimuli, the device comprising:
a plurality of electrodes including one or more stimulus electrodes and one or more measurement electrodes;
a stimulus source configured to provide neural stimuli to be delivered via the one or more stimulus electrodes to a neural pathway of a patient in order to evoke a neural response from the neural pathway;
measurement circuitry configured to capture signal windows sensed on the neural pathway via the one or more measurement electrodes subsequent to respective neural stimuli; and
a control unit configured to control the stimulus source to provide each neural stimulus according to a stimulus intensity parameter;
a processor configured to:
instruct the control unit to control the stimulus source to provide a neural stimulus according to a stimulus intensity parameter; and
measure an intensity of an evoked neural response in a signal window captured subsequent to the provided neural stimulus by correlating the captured signal window with a template,
wherein the template is orthogonal to a predetermined artefact basis that models an artefact component in the captured signal window.

12. The system of claim 11, wherein the processor is further configured to:
determine a feedback variable from the measured intensity of the evoked neural response; and
adjust, using a feedback controller, the stimulus intensity parameter so as to maintain the feedback variable at a target value.

13. The system of claim 2, wherein the processor is further configured to derive the template based on a comparison between new therapy parameters and therapy parameters according to which the neural stimulus was provided.

14. The system of claim 13, wherein the processor is configured to derive the template by:
projecting a representative signal comprising an evoked neural response component onto the predetermined artefact basis; and
deriving the template by subtracting the projected representative signal from the representative signal.

15. The system of claim 14, wherein the processor is further configured to obtain the representative signal from one or more signal windows captured subsequent to respective stimuli delivered using the new therapy parameters.

16-37. (canceled)
